(19) United States
(12) Patent Application Publication
(10) Pub. No.: US 2023/0215548 A1
(43) Pub. Date: Jul. 6, 2023

(54) SELF ASSIGNMENT WITH AUTOMATIC DETECTION OF MULTIPLE CAREGIVERS

(71) Applicant: Rauland-Borg Corporation, Mount Prospect, IL (US)

(72) Inventors: Gina Pampalone Brody, Mount Prospect, IL (US); Paul Duda, Mount Prospect, IL (US); Karthik Srivathsa, Mount Prospect, IL (US); Steve Grabowski, Mount Prospect, IL (US); Timothy Keller, Mount Prospect, IL (US)

(73) Assignee: Rauland-Borg Corporation, Mount Prospect, IL (US)

(21) Appl. No.: 17/569,213

(22) Filed: Jan. 5, 2022

(51) Int. Cl. G16H 40/20; G16H 10/60

(52) U.S. Cl. CPC G16H 40/20; G16H 10/60

(57) ABSTRACT

A caregiver converge device and system associated with a patient room within a hospital are provided. The device and system include: an operating system configured to provide operational states of the caregiver converge device, the operational states provide work flow functionality for an assigned caregiver of a patient in the patient room; a touch screen display for displaying a user interface configured to facilitate the assigned caregiver utilizing the work flow functionality, the touch screen display configured to accept input from the assigned caregiver; and a caregiver proximity sensor configured to determine whether the assigned caregiver enters the patient room; where the operating system updates the work flow functionality for the assigned caregiver upon the caregiver proximity sensor determining the assigned caregiver enters the patient room.

### BACKGROUND OF THE INVENTION

Typically, a hospital includes various units that service patients of the hospital. A non-exhaustive list of units within a hospital may include: an Intensive Care Unit (ICU), a medical/surgery unit, a maternity ward, and an Emergency Department (ED). Further, each unit within the hospital typically includes a variety of patient rooms.

A hospital typically employs many caregivers tasked with providing services to the patients. Typically, these caregivers are both highly skilled and trained to provide a specialized care for the patients. Given the unique needs/conditions of each patient, the caregivers are typically assigned based on an ability to provide services to the patient. Further, given that hospitals typically operate twenty-four hours a day, every day of the year, a large number of caregivers are needed to cover several shifts taking care of patients throughout the day.

In view of the above, hospitals typically make use of complicated administrative systems to track which caregivers are assigned to different units within the hospital, and, of those caregivers, which caregivers are assigned to a particular patient or patient room. Current administrative systems to track caregivers are inefficient.

### BRIEF SUMMARY OF THE INVENTION

The disclosure provides a caregiver converge device associated with a patient room within a hospital. The caregiver converge device includes: an operating system configured to provide operational states of the caregiver converge device, the operational states provide work flow functionality for an assigned caregiver of a patient in the patient room; a touch screen display for displaying a user interface configured to facilitate the assigned caregiver utilizing the work flow functionality, the touch screen display configured to accept input from the assigned caregiver; and a caregiver proximity sensor configured to determine whether the assigned caregiver enters the patient room; where the operating system updates the work flow functionality for the assigned caregiver upon the caregiver proximity sensor determining the assigned caregiver enters the patient room..

The disclosure further provides a caregiver converge system for integration in a plurality of patient rooms within a hospital. The caregiver converge system includes: a caregiver assignment server configured to manage caregiver patient room assignments; and a plurality of caregiver converge devices associated with the plurality of patient rooms on a one-to-one basis; where each caregiver converge device of the plurality of caregiver converge devices includes: a network interface connecting the caregiver converge device with the caregiver assignment server; an operating system configured to provide operational states of the caregiver converge device, the operational states provide work flow functionality for an assigned caregiver of a patient in the patient room; a touch screen display for displaying a user interface configured to facilitate the assigned caregiver utilizing the work flow functionality, the touch screen display configured to accept input from the assigned caregiver; and a caregiver proximity sensor configured to determine whether the assigned caregiver enters the patient room; where the operating system updates the work flow functionality for the assigned caregiver upon the caregiver proximity sensor determining the assigned caregiver enters the patient room.

Additionally, the disclosure provides a caregiver converge device associated with a patient room within a hospital. The caregiver converge device includes: a network interface connecting the caregiver converge device with a caregiver assignment server, the caregiver assignment server configured to manage caregiver assignments to the patient room; an operating system configured to provide operational states of the caregiver converge device, the operational states provide work flow functionality for an assigned caregiver of a patient in the patient room; a touch screen display for displaying a user interface configured to facilitate the assigned caregiver utilizing the work flow functionality, the touch screen display configured to accept input from the assigned caregiver; and a caregiver proximity sensor configured to determine whether the assigned caregiver enters the patient room; where the operating system updates the work flow functionality for the assigned caregiver upon the caregiver proximity sensor determining the assigned caregiver enters the patient room, where the assigned caregiver is a plurality of assigned caregivers for providing services to the patient in the patient room; where the caregiver proximity sensor is further configured to determine an identity of a specific assigned caregiver of the plurality of assigned caregivers upon the specific assigned caregiver entering the patient room; where the operating system updates the work flow functionality for the specific assigned caregiver based on the determined identity, and where the work flow functionality is different for each assigned caregiver of the plurality of assigned caregivers.

### BRIEF DESCRIPTION OF THE SEVERAL VIEWS OF THE DRAWING(S)

FIG. 1 illustrates a hospital Information Technology (IT) network, in accordance with an embodiment of the disclosure;

FIG. 2 illustrates nurse call equipment, in accordance with an embodiment of the disclosure;

FIG. 3 illustrates an exemplary arrangement of nurse call equipment within a patient room, in accordance with an embodiment of the disclosure;

FIG. 4 illustrates a caregiver converge device, in accordance with an embodiment of the disclosure;

FIG. 5 illustrates aspects of the caregiver converge device of FIG. 4;

FIG. 6 illustrates further aspects of the caregiver converge device of FIG. 4;

FIGS. 7A and 7B illustrate further aspects of the caregiver converge device of FIG. 4;

FIG. 8 illustrates a caregiver change performed at the caregiver device of FIG. 4, in accordance with an embodiment of the disclosure;

FIG. 9 illustrates a method for caregiver change, in accordance with an embodiment of the disclosure;

FIG. 10 illustrates further aspects of the caregiver converge device of FIG. 4; and

FIG. 11 illustrates an electronic medical record, in accordance with an embodiment of the disclosure.

### DETAILED DESCRIPTION OF THE INVENTION

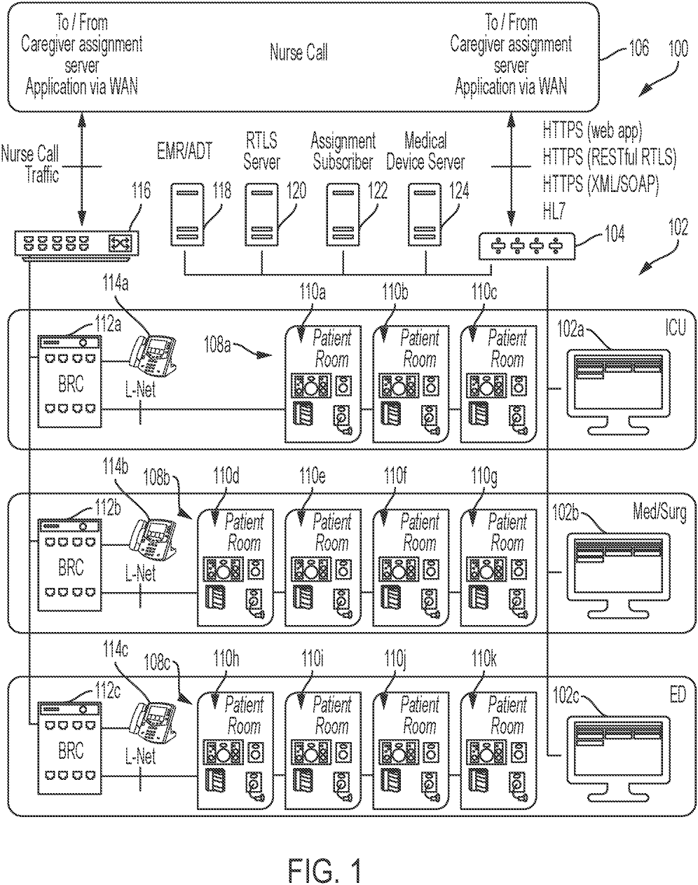

FIG. 1 illustrates an exemplary hospital Information Technology (IT) network 100. Hospital IT network 100 includes hospital units 102, including an Intensive Care Unit (ICU) 102a, a medical/surgery unit 102b, and an Emergency Department (ED) 102c. Each of the hospital units 102 are networked via a router 104 with a nurse call and caregiver assignment publisher server 106. The nurse call and caregiver assignment publisher server 106 manages nurse call equipment and caregiver assignments for each of the hospital units 102.

Additionally, the hospital IT network 100 includes further IT sub-servers including an Electronic Medical Records/Admission, Transfer (EMR/ADT) server 118, a Real Time Location System (RTLS) server 120, an Assignment Subscriber server 122, and a Medical Device server 124. The EMR/ADT server 118 keeps and provides access to medical records of patients within the hospital for caregivers to review and/or update those records accordingly. The RTLS server 120 enables an RTLS system within the hospital that tracks individual caregiver locations such that the RTLS server 120 can provide a location of any individual caregiver at any time while that caregiver is in the hospital. The Assignment Subscriber server 122 allows caregivers and equipment to subscribe to message flow for various hospital units. The Medical Device server 124 provides network functionality for medical devices within the hospital. Each of the EMR/ADT server 118, the RTLS server 120, the Assignment Subscriber server 122, and the Medical Device server 124 are communicatively coupled with the nurse call and caregiver assignment publisher server 106 via the router 104, in the illustrated embodiment.

Each hospital unit 102a, 102b, and 102c include respective patient rooms 108a, 108b, and 108c. The patient rooms 108a, 108b, and 108c include nurse call equipment 110a-110k. As shown in the illustrated embodiment, nurse call equipment 110a-110k is the same in each patient room. However, in other embodiments, nurse call equipment 110a-110k can be different equipment in each room and is dependent on a hospital preferred installation.

Each hospital unit 102a, 102b, and 102c also include a respective administrative console 114a, 114b, and 114c. The administrative consoles 114a, 114b, and 114c provide a centralized area where administrative functions are performed by an administrator for each of the hospital units 102a, 102b, and 102c, respectively. For each hospital unit 102a, 102b, and 102c, the administrative consoles 114a, 114b, and 114c along with the nurse call equipment 110a-110k are connected to a router 116 via Brach Regional Controllers (BRCs) 112a, 112b, and 112c. The router 116 networks the administrative consoles 114a, 114b, and 114c along with the nurse call equipment 110a-110k to the nurse call and caregiver assignment publisher server 106.

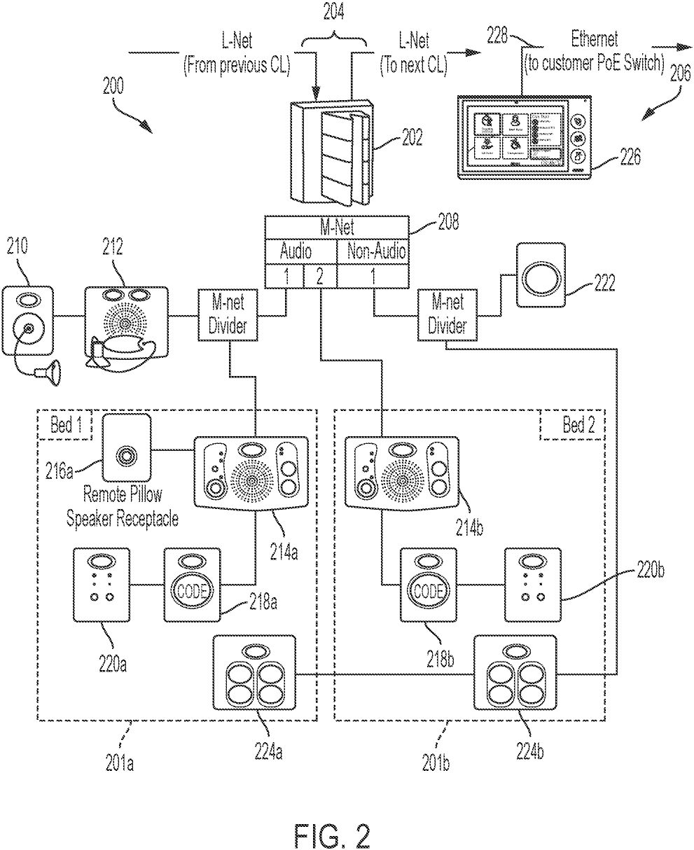

FIG. 2 illustrates an embodiment of a patient room 200 including beds 201a and 201b and nurse call equipment 206. The nurse call equipment 206 includes a corridor light 202 that provides a visual room status. The corridor light 202 is networked with the nurse call and caregiver assignment publisher server 106 (please see FIG. 1, not shown in FIG. 2) via network connection 204. The corridor light 202 is connected to a network connector 208. The network connector 208 includes audio ports 1 and 2 and non-audio port 1.

Audio port 1 of the network connector 208 connects room pull cord stations 210 and 212 to the corridor light 202. Audio port 1 further connects the corridor light 202 with a pillow speaker 214a and its remote pillow speaker receptacle 216a for bed 201a. Audio port 1 further connects the corridor light 202 with a Code Blue pushbutton Station 218a and a dual ¼ inch jack station 220a of bed 201a. The Code Blue pushbutton Station 218a allows a Code Blue event to be triggered with respect to bed 201a, and the dual ¼ inch jack station 220a allows for various auxiliary call placement devices to connect and place a call.

Audio port 2 connects the corridor light 202 with similar structure but for bed 210b. In the illustrated embodiment, this structure includes pillow speaker 214b that in turn connects to a Code Blue pushbutton Station 218b and a dual ¼ inch jack station 220b. The pillow speaker 214b, Code Blue pushbutton Station 218b, and dual ¼ inch jack station 220 operate similar to their corresponding structure from bed 201a. Specifically, the Code Blue pushbutton Station 218b allows a Code Blue event to be triggered with respect to bed 201b, and the dual ¼ inch jack station 220b allows for various auxiliary call placement devices to connect and place a call.

Further, non-audio port 1 connects the corridor light 202 with bedside push button stations 224a and 224b for beds 201a and 201b, respectively. Additionally, non-audio port 1 connects the corridor light 202 to a room emergency button 222. Signaling for all of the aforementioned nurse call equipment with respect to the patient room 200 is routed through the corridor light 202 to the nurse call and caregiver assignment server (not shown) for processing by the further hospital IT system 100 (see FIG. 1).

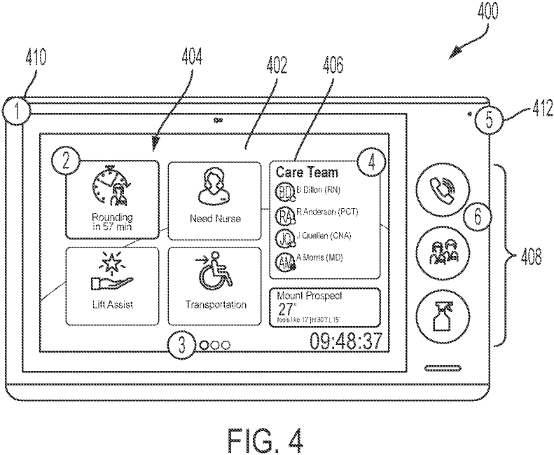

Additionally, nurse call equipment 206 of patient room 200 further includes a caregiver converge device 226 that connects to the nurse call and caregiver assignment server (not shown) via a network connection 228, which is shown as a Power over Ethernet (PoE) connection in the illustrated embodiment. The caregiver converge device 226 facilitates caregiver activities within the patient room 200, and its structure and functionality is be more fully described in view of FIGS. 4-11.

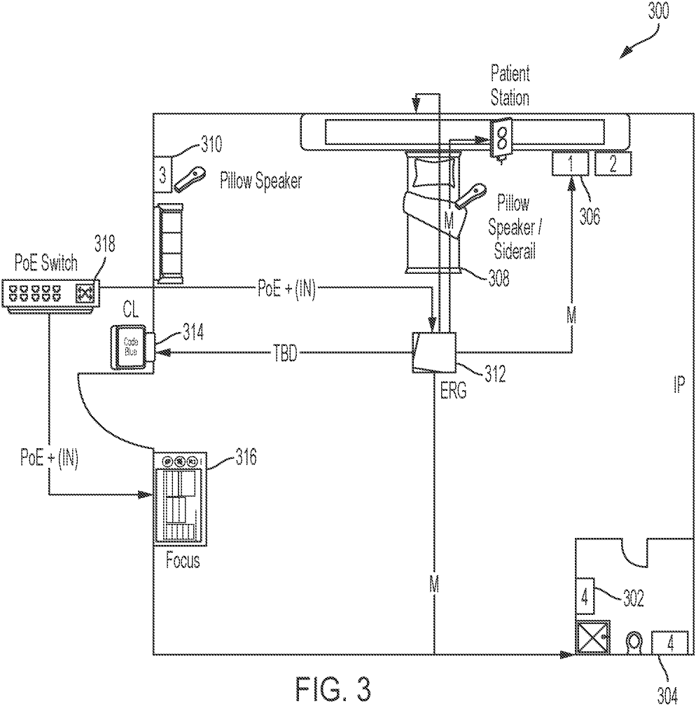

FIG. 3 illustrates a layout of a patient room 300, in accordance with an embodiment of the disclosure. Patient room 300 includes pull cord stations 302 and 304 arranged in a bathroom of the patient room 300. Patient room 300 further includes a button station 306 and pillow speaker 310 associated with a patient bed 308. Each of the pull cord stations 302 and 304, the button station 306, the pillow speaker 310 and the patient bed 308 are networked via an ERG 312 to a corridor light 314 for connection to the nurse call and caregiver assignment server (not shown).

Further, patient room 300 includes a caregiver converge device 316 communicatively coupled with the ERG 312 via a switch 318, which in the illustrated embodiment is a PoE switch 318. The caregiver converge device 316 enables efficient caregiver functionality for a patient within patient room 300. As illustrated, the caregiver converge device 316 is located proximate an entrance to the patient room 300. By locating the caregiver converge device 316 close to the entrance, a caregiver is able to access the caregiver functionality provided by the caregiver converge device 316 easily without having to traverse is a location within the room. This provides increased efficiency in that it enables a caregiver to complete a task multiple times for multiple patient rooms without having to take excess steps to accomplish the task in each patient room.

Moreover, while FIGS. 3 and 4 illustrate embodiments of the disclosure providing only a single patient room, additional patient rooms with additional nurse call equipment is contemplated. For instance, as shown in FIG. 1, a typical hospital includes many patient rooms. In certain embodiments, each patient room will have associated nurse call equipment, including a caregiver converge device. In each patient room, the caregiver converge device will operate similarly. Such functionality is described below for a single caregiver converge device, but it is contemplated that this functionality can be individually applied to a plurality of caregiver converge device interfacing with a plurality of caregivers attending to patients within a plurality of patient rooms within the hospital.

FIG. 4 illustrates a caregiver converge device 400, in accordance with certain embodiments. The caregiver converge device 400 includes a touchscreen display 402 that provides a user interface for use by a caregiver within a hospital. The user interface allows a caregiver interacting with the caregiver converge device 400 to interact with an operating system running on the caregiver converge device 400. The operating system provides workflow functionality associated with the caregiver through the caregiver converge device 400. The workflow functionality is provided through the caregiver converge device 400 by the operating system communicatively interacting with the nurse call and caregiver assignment publisher server 106 (see FIG. 1) to update the information contained and managed at the server. In this fashion, the operating system of the caregiver converge device 400 provides the associated workflow functionality customized for a specific caregiver at the caregiver converge device 400. While not illustrated in FIG. 4, the caregiver converge device 400 includes at least one processor and at least one memory. The memory stores program code of the operating system that is executed by the processor in order to provide the workflow functionality at the caregiver converge device 400 for assigned caregivers.

In the illustrated embodiment, the user interface displays a home screen of the caregiver converge device 400. The home screen displays workflow buttons 404 that enable a caregiver utilizing the caregiver converge device 400 to access workflow functionality facilitated by the caregiver converge device 400. In the illustrated embodiment, the workflow buttons 404 include a “Rounding” button, a “Need Nurse” button, a “Lift Assist” button, and a “Transportation” button. The “Rounding” button enables a caregiver to update and log performance of rounds for a particular patient room where the caregiver converge device 400 is located. In certain embodiments, the “Rounding” button also displays a countdown timer set to provide a time when the caregiver will next perform the rounds in the patient room. The “Need Nurse” button that will page a nurse caregiver to the patient room when the button is pressed, and the “Transportation” button will page specific caregivers assigned to transport a patient in the patient room including the caregiver converge device 400.

The home screen of the user interface for the caregiver converge device 400 further includes a list of caregivers 406 assigned to the patient in the patient room associated with the caregiver converge device 400. In this manner, a caregiver can quickly scan the home screen of the caregiver converge device 400 and determine caregivers associated with the patient in the patient room.

The caregiver converge device 400 further includes quick access buttons 408. The quick access buttons 408 enable a caregiver to quickly access functionality of the caregiver converge device 400 without having to navigate to a particular screen of the user interface on the touchscreen display 402. In the illustrated embodiment, the quick access buttons 408 include a button for placing a two-way audio call through the caregiver converge device 400 to a preconfigured phone extension, a button for requesting all assigned caregiver staff for the patient come to the patient room associated with the caregiver converge device 400, and a button for requesting environmental cleanup services in the patient room associated with the caregiver converge device 400.

In addition to the quick access buttons 408, the caregiver converge device 400 further includes a cancel button 410, which is another hardware button. In the illustrated embodiment, the cancel button 410 is located on a top of the caregiver converge device 400 and configured to end specific nurse call alerts from the quick access buttons 408 or end other routine/workflow nurse call functionality pertinent to the patient room associated with the caregiver converge device 400. In certain embodiments, the cancel button 410 only functions to end nurse call activity within the patient room or routine/workflow functionality for other in-room, non-patient stations.

The caregiver converge device 400 further includes a proximity sensor 412. The proximity sensor 412 is configured to detect that a caregiver has entered the patient room associated with the caregiver converge device 400. In certain embodiments, the proximity sensor 412 detects a RTLS badge carried by the caregiver and interfaces with the RTLS server 120 (see FIG. 1) to determine proximity of the caregiver. The RTLS proximity sensing occurs through wireless communication to detect when a caregivers’ RTLS badge is within wireless range of the caregiver converge device 400. In this manner, a unique badge number for each caregiver would be entered in a database associated with the RTLS server 120 (see FIG. 1), and the caregiver converge device 400 detects the unique badge number and transmits it to the RTLS server 120. The RTLS server 120 then provides associated caregiver information to the caregiver converge device 400.

In other embodiments, the proximity sensor 412 can detect a proximity (prox) card carried by the caregiver, such as an Identification (ID) card of the caregiver. In this manner, when the caregiver walks into the patient room with an RTLS badge, or places their ID card on the proximity sensor 412, the caregiver converge device 400 determines whether that caregiver is currently assigned to the patient room, and, if so, configures the workflow functionality of the caregiver converge device 400 to provide functionality specific to the detected caregiver. In this manner, a caregiver is able to seamlessly access workflow functionality specific to their assigned duties at the caregiver converge device 400 without having to specifically and manually log in to a device or change their access with an administrator. This improves efficiency for performing the workflow associated with the caregiver.

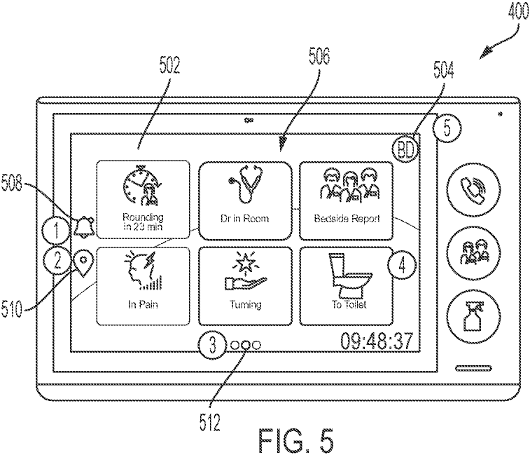

FIG. 5 illustrates updated functionality provided on a staff workflow screen 502 of the user interface of the caregiver converge device 400 upon a caregiver (i.e., nurse) entering the patient room. Upon detection of the caregiver, the caregiver converge device 400 updates a staff icon 504 to display initials of the caregiver along with the role specific workflow. This workflow and initials will be updated to other assigned caregivers once they enter the patient room, similar to the illustrated embodiment.

The staff workflow screen 502 further illustrates role-specific staff workflow buttons 506. In the illustrated embodiment, buttons 506 include a “Rounding” button, a “Dr in Room” button, a “Bedside Report” button, an “In Pain” button, a “Turning” button, and a “In Toilet” button. Similar to the previous description, the “Rounding” button enables the caregiver to update and log performance of rounds for the patient room where the caregiver converge device 400 is located. In certain embodiments, the “Rounding” button also displays a countdown timer set to provide a time when the caregiver will next perform the rounds in the patient room. The “Dr in Room” button may be pressed by a physician (i.e., caregiver) upon entering the room to update the status of the patient room to indicate that a doctor is in the room attending to the patient. In certain embodiments, this will instruct the associated nurse call system to indicate that a doctor is in the patient room. The “Bedside Report” button, when pressed, indicates that bedside reporting is taking place in the room. In certain embodiments, such an event may be noted in the EMR. Bedside reporting happens at the change of shift, outgoing shift, and incoming shift when the caregiver meets with the patient and/or patient’s family to discuss what happened and what is planned. The “In Pain” button enables the caregiver to quickly update the nurse call system that the patient is in pain and that mitigating procedures should be taken to alleviate the pain. The “Turning” button provides notice that a patient needs to be turned because the patient is unable to turn themselves while in bed. The “To Toilet” button allows the patient to indicate that the patient needs assistance from a caregiver to use the toilet.

The caregiver converge device 400 further includes an alert list icon 508 and a team location icon 510. When a caregiver presses the alert list icon 508 a list of all active nurse call alerts that are assigned to the caregiver is provided on the touchscreen display. When the caregiver presses the team location icon 510, the caregiver converge device 400 displays a location of all caregivers currently assigned to the patient room. In certain embodiments, the location is determined from the RTLS server 120 (see FIG. 1) or from the nurse call and caregiver assignment publisher server 106, as updated via use of prox cards carried by the associated caregivers.

The caregiver converge device 400 in FIG. 5 further illustrates a page indicator 512. In certain embodiments, a caregiver accessing the caregiver converge device 400 may have multiple pages of role-specific staff workflow buttons 506. The caregiver can cycle through pages containing the buttons 506 by making a swipe gesture of the touchscreen display. The page indicator 512 both illustrates a number of such pages and a location of the current page displayed within the available pages. In certain embodiments, the swipe gesture may be a left to right swipe or a right to left swipe. In alternative embodiments, the swipe gesture may be up or down, or in any other direction.

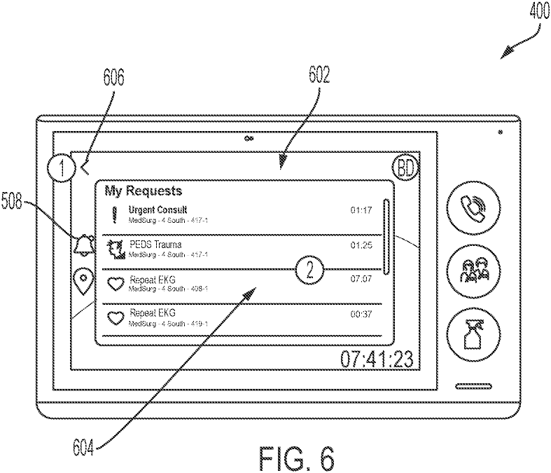

FIG. 6 illustrates another embodiments of the caregiver converge device 400. In the illustrated embodiment, the touchscreen display of the caregiver converge device 400 shows a “My Requests” screen 602 of the user interface. This screen is shown after the caregiver presses the alert list icon 508 (see FIG. 5). The “My Requests” screen 602 shows open calls and notifications 604 relevant to the detected assigned caregiver and the associated patient. In certain embodiments, urgent calls, such as a code call will also be displayed on the “My Requests” screen 602. The caregiver is able to navigate back to the role-specific staff workflow screen 506 (see FIG. 5) using a back button 606.

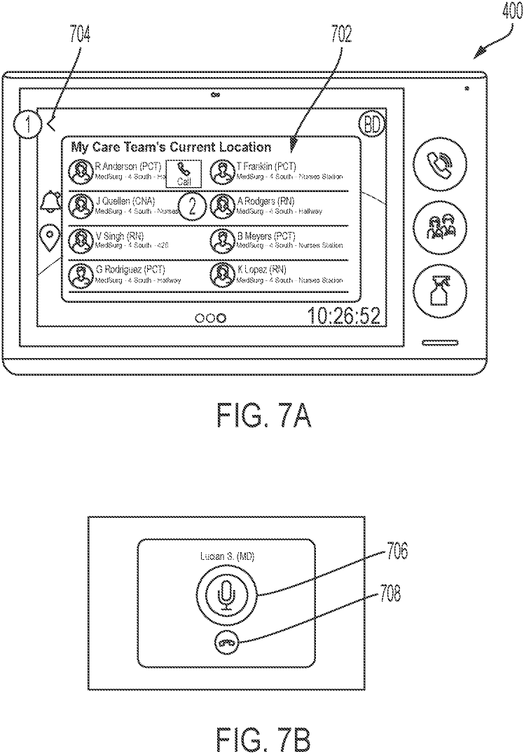

FIG. 7A illustrates another embodiment of the caregiver converge device 400. In the illustrated embodiment, the touchscreen display of the caregiver converge device 400 shows a caregiver team current location screen 702 of the user interface. The caregiver team current location screen 702 shows a list of assigned caregiver location within the hospital. The location is determined form the RTLS server 120 (see FIG. 1) or via the nurse call and caregiver assignment publisher server 106. The caregiver current location screen 702 is shown after a caregiver presses the team location icon 510 (see FIG. 5). The caregiver is able to navigate back to the role-specific staff workflow screen 506 using a back button 704.

FIG. 7B illustrates a call button 706 that when pressed calls a selected caregiver. The caregiver is selected from the caregiver team current location screen 702 by an assigned caregiver selecting another assigned caregiver from the location screen 702 by performing a specific gesture, such as pressing or swiping, the name in the location screen 702. Upon performing the gesture, the user interface displays the call button 706 that if pressed will place a call to the selected assigned caregiver from the caregiver converge device 400. Additionally, an end call button 708 is displayed for ending the call to the selected assigned caregiver.

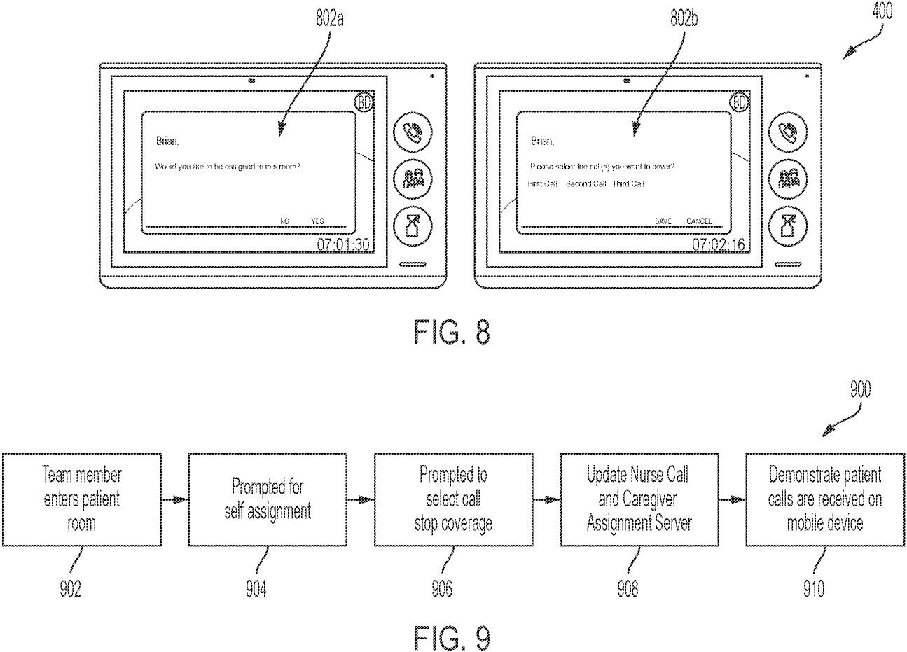

FIG. 8 illustrates embodiments of the user interface during a caregiver assignment based on proximity to the caregiver converge device 400. Upon detection of a new caregiver (i.e., not currently assigned to provide care for any patient in the patient room associated with caregiver converge device 400) entering the patient room via the proximity sensor 412 (see FIG. 4), the caregiver converge device 400 configures the user interface to display a prompt for assignment screen 802a. The prompt for assignment screen 802a asks the detected, newly entered and not yet assigned caregiver whether they would like to be assigned as a caregiver to the patient room. If the caregiver selects to be assigned to the patient room in the assignment screen 802a, the caregiver converge device 400 then displays a call stop selection screen 802b. The call stop selection screen 802b enables the newly assigned caregiver to select which call alert they would like to receive for calls from the patient room associated with the caregiver converge device 400. In the illustrated embodiment, the newly assigned caregiver can select receiving first, second, or third call alerts. Please note the illustrated embodiment is one embodiment, and, in further embodiments, more or less than three call alerts are contemplated. After the selection of call alerts, the newly assigned caregiver can then press save on the call stop selection screen 802b, and the caregiver converge device 400 updates the nurse call and caregiver assignment publisher server 106 with the updated caregiver information for the patient room. In this manner, a caregiver can be newly assigned to a patient room directly by the caregiver upon entering the patient room and being detected by the newly assigned to a patient room directly by the caregiver upon entering the patient room and being detected by the proximity sensor 412 of the caregiver converge device 400. This improves efficiency of the nurse call system by providing for automatic on location assignments without having to manually make the assignment change at a central console or alert administrative personnel to make the change to the nurse call system manually. Further, this facilitates quick assignment of a caregiver to a plurality of patient rooms when that caregiver comes on shift at the hospital.

FIG. 9 illustrates a flow chart 900 of steps performed by the caregiver converge device 400 (see FIG. 8) during the assignment of a new caregiver. At step 902, the caregiver converge device 400 detects a newly entered and not yet assigned caregiver. At step 904, the caregiver converge device 400 prompts the newly entered and not yet assigned caregiver for performing a self assignment to the patient room. After the newly entered and not yet assigned caregiver selects to be assigned to the patient room, at step 906, the caregiver converge device 400 prompts the newly assigned caregiver to select call stop coverage, and, at step 908, updates the nurse call and caregiver assignment publisher server 106 (see FIG. 1) with the newly assigned caregiver’s information for the patient room. At step 910, the caregiver converge device 400 initiates a call the newly assigned caregiver via a mobile device carried by the newly assigned caregiver in order to demonstrate that the selected call stop functions properly.

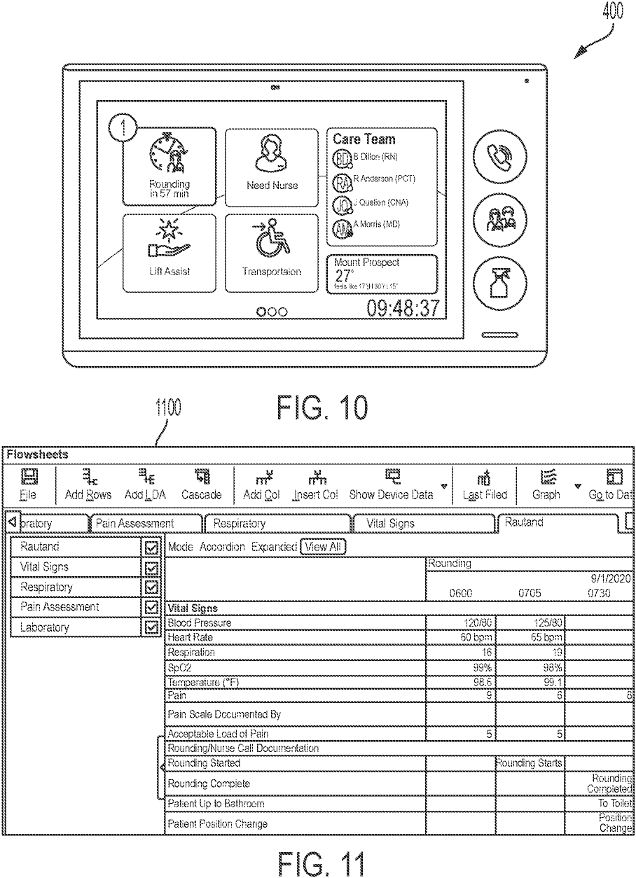

FIGS. 10 and 11 illustrate workflow functionality of the caregiver converge device 400 for performing EMR charting through the caregiver converge device 400. This workflow functionality captures relevant information regarding a patient during caregiver interaction with the patient at the caregiver converge device 400 and updates the patient’s EMR 1100 at the EMR/ADT server 118. In this regard, the caregiver converge device 400 is the entry point for values that can be sent to the EMR system 118 (see FIG. 1) from a device in the room. Values are sent via HL7 and are typically saved in a patient flowsheet.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A caregiver converge device associated with a patient room within a hospital, the caregiver converge device comprising:
an operating system configured to provide operational states of the caregiver converge device, the operational states provide work flow functionality for an assigned caregiver of a patient in the patient room;
a touch screen display for displaying a user interface configured to facilitate the assigned caregiver utilizing the work flow functionality, the touch screen display configured to accept input from the assigned caregiver; and
a caregiver proximity sensor configured to determine whether the assigned caregiver enters the patient room;
wherein the operating system updates the work flow functionality for the assigned caregiver upon the caregiver proximity sensor determining the assigned caregiver enters the patient room.

2. The caregiver converge device of claim 1, wherein the assigned caregiver is a plurality of assigned caregivers for providing services to the patient in the patient room;
wherein the caregiver proximity sensor is further configured to determine an identity of a specific assigned caregiver of the plurality of assigned caregivers upon the specific assigned caregiver entering the patient room; and
wherein the operating system updates the work flow functionality for the specific assigned caregiver based on the determined identity.

3. The caregiver converge device of claim 2, wherein the work flow functionality is different for each assigned caregiver role of the plurality of assigned caregivers.

4. The caregiver converge device of claim 2, wherein the operational states comprise a caregiver location state that configures the touch screen display to show a current location of each of the plurality of assigned caregivers within the hospital.

5. The caregiver converge device of claim 4, further comprising a network interface connecting the caregiver converge device with a Real Time Location System (RTLS);
wherein the caregiver proximity sensor receives location information for the plurality of assigned caregivers from the RTLS.

6. The caregiver converge device of claim 1, further comprising a network interface connecting the caregiver converge device with an Information Technology (IT) network of the hospital.

7. The caregiver converge device of claim 6, wherein the network interface is communicatively connected to a caregiver assignment server of the IT network, the caregiver assignment server being configured to manage caregiver patient room assignments;
wherein upon the caregiver proximity sensor determining that a new caregiver enters the patient room that is not the assigned caregiver, the operating system is configured to:
prompt the new caregiver for self-assignment to the patient room;
prompt for call stop information;
update the caregiver assignment server tracking assigned caregivers for the patient room to include the new caregiver; and
demonstrate patient calls for the patient in the patient room are received on a mobile device associated with new caregiver.

8. The caregiver converge device of claim 6, wherein the network interface is communicatively connected with an Electronic Medical Records (EMR) system of the IT network;
wherein the operating system sends an update regarding a patient record to the EMR system; and
wherein the update comprises events associated with the work flow functionality for the assigned caregiver.

9. The caregiver converge device of claim 6, further comprising:
a speaker;
a microphone; and
a physical push button separate from the touchscreen display;
wherein, when the physical push button is pressed, the operating system places a two-way audio call to a preconfigured extension within the hospital.

10. The caregiver converge device of claim 1, wherein the caregiver proximity sensor is a proximity card reader configured to detect an identification card of the assigned caregiver when the identification card is proximate the caregiver proximity sensor.

11. A caregiver converge system for integration in a plurality of patient rooms within a hospital, the caregiver converge system comprising:
a caregiver assignment server configured to manage caregiver patient room assignments; and
a plurality of caregiver converge devices associated with the plurality of patient rooms on a one-to-one basis;
wherein each caregiver converge device of the plurality of caregiver converge devices comprises:
a network interface connecting the caregiver converge device with the caregiver assignment server;
an operating system configured to provide operational states of the caregiver converge device, the operational states provide work flow functionality for an assigned caregiver of a patient in the patient room;
a touch screen display for displaying a user interface configured to facilitate the assigned caregiver utilizing the work flow functionality, the touch screen display configured to accept input from the assigned caregiver; and
a caregiver proximity sensor configured to determine whether the assigned caregiver enters the patient room;
wherein the operating system updates the work flow functionality for the assigned caregiver upon the caregiver proximity sensor determining the assigned caregiver enters the patient room.

12. The caregiver converge system of claim 11, wherein the assigned caregiver is a plurality of assigned caregivers for providing services to the patient in the patient room;
wherein the caregiver proximity sensor is further configured to determine an identity of a specific assigned caregiver of the plurality of assigned caregivers upon the specific assigned caregiver entering the patient room; and
wherein the operating system updates the work flow functionality for the specific assigned caregiver based on the determined identity.

13. The caregiver converge system of claim 12, wherein the work flow functionality is different for each assigned caregiver of the plurality of assigned caregivers.

14. The caregiver converge system of claim 12, wherein the operational states comprise a caregiver location state that configures the touch screen display to show a current location of each of the plurality of assigned caregivers within the hospital.

15. The caregiver converge system of claim 14, wherein the network interface communicatively connects the caregiver converge device with a Real Time Location System (RTLS);
wherein the caregiver proximity sensor receives location information for the plurality of assigned caregivers from the RTLS.

16. The caregiver converge system of claim 11, wherein upon the caregiver proximity sensor determining that a new caregiver enters the patient room that is not the assigned caregiver, the operating system is configured to:
prompt the new caregiver for self-assignment to the patient room;
prompt for call stop information;
update the caregiver assignment server tacking assigned caregivers for the patient room to include the new caregiver; and
demonstrate patient calls for the patient in the patient room are received on a mobile device associated with new caregiver.

17. The caregiver converge system of claim 11, wherein the network interface is communicatively connected with an Electronic Medical Records (EMR) system;
wherein the operating system sends an update regarding a patient record to the EMR system; and
wherein the update comprises events associated with the work flow functionality for the assigned caregiver.

18. The caregiver converge system of claim 11, wherein each caregiver converge device of the plurality of caregiver converge devices further comprises:
a speaker;
a microphone; and
a physical push button separate from the touchscreen display;
wherein, when the physical push button is pressed, the operating system places a two-way audio call to a preconfigured extension within the hospital.

19. The caregiver converge system of claim 11, wherein the caregiver proximity sensor is a proximity card reader configured to detect an identification card of the assigned caregiver when the identification card is proximate the caregiver proximity sensor.

20. A caregiver converge device associated with a patient room within a hospital, the caregiver converge device comprising:
a network interface connecting the caregiver converge device with a caregiver assignment server, the caregiver assignment server configured to manage caregiver assignments to the patient room;
an operating system configured to provide operational states of the caregiver converge device, the operational states provide work flow functionality for an assigned caregiver of a patient in the patient room;
a touch screen display for displaying a user interface configured to facilitate the assigned caregiver utilizing the work flow functionality, the touch screen display configured to accept input from the assigned caregiver; and
a caregiver proximity sensor configured to determine whether the assigned caregiver enters the patient room;
wherein the operating system updates the work flow functionality for the assigned caregiver upon the caregiver proximity sensor determining the assigned caregiver enters the patient room,
wherein the assigned caregiver is a plurality of assigned caregivers for providing services to the patient in the patient room;
wherein the caregiver proximity sensor is further configured to determine an identity of a specific assigned caregiver of the plurality of assigned caregivers upon the specific assigned caregiver entering the patient room;
wherein the operating system updates the work flow functionality for the specific assigned caregiver based on the determined identity, and
wherein the work flow functionality is different for each assigned caregiver of the plurality of assigned caregivers.
